(19) United States
(12) Patent Application Publication
(10) Pub. No.: US 2023/0372686 A1
(43) Pub. Date: Nov. 23, 2023

(54) APPARATUS AND METHOD FOR DILATING A BLOOD VESSEL AND SUBCUTANEOUS TISSUE

(71) Applicant: J & M SURGICAL, OAK PARK, IL (US)

(72) Inventors: Asif K. MUSTAFA, Willowbrook, IL (US); Devang J. Joshi, Carol Stream, IL (US)

(21) Appl. No.: 17/584,542

(22) Filed: Jan. 26, 2022

(51) Int. Cl. A61M 29/02; A61M 25/01

(52) U.S. Cl. CPC A61M 29/02; A61M 25/0108; A61M 2029/025; A61M 2025/09133

(57) ABSTRACT

This invention relates to an apparatus and method for the dilation of a blood vessel and subcutaneous tissue that eliminates or minimizes a successive and repetitive insertion and removal of multiple, individual dilators necessary to achieve a target dilation. The dilator preferably defines radiopaque markers to make them visible to a medical practitioner via fluoroscopy. A hydrophilic coating is optionally located on the dilator to reduce surface friction and enhance lubricity between the dilator and vessel and/or subcutaneous tissue.

### CROSS REFERENCE TO RELATED APPLICATIONS

This non-provisional patent application claims priority to U.S. Provisional Patent Application Ser. No. 63/257,070 having a filing date of Oct. 18, 2021, which is fully incorporated by reference herein.

### TECHNICAL FIELD OF THE INVENTION

This invention relates generally to dilators used in endovascular medical procedures. More specifically, the invention relates to an apparatus and method for the dilation of a blood vessel and subcutaneous tissue that eliminates or minimizes a successive insertion and removal of multiple, individual dilators necessary to achieve a target dilation.

### BACKGROUND OF THE INVENTION

Endovascular dilators are used to pre-dilate blood vessels and subcutaneous tissue that must be navigated for the delivery and placement of endovascular devices, such as catheters or cannulas. Arterial cannulation, for example, is frequently performed in an acute and critical care setting to accurately measure blood and arterial pressures. The pressure measurements allow for immediate recognition of clinical alterations to allow for the ready intervention and stabilization of a patient. Cannulation is also used to connect a patient to the tubing of a cardiopulmonary bypass (CBP) or extracorporeal membrane oxygenation (ECMO) machine to ensure the proper oxygenation of blood during surgical (i.e., open heart surgery) or extended critical care (i.e., ICU) procedures.

For the placement of a cannula, a “cut-down” procedure using a scalpel and electrocautery may be utilized to expose a target blood vessel to be dilated, after which, a needle is used to puncture the vessel. Alternatively, a needle may be utilized to percutaneously puncture the blood vessel without a cutdown. After the blood vessel is punctured with the needle, the forward end of a flexible guide wire is inserted through the needle, into the vessel and advanced by an appropriate distance to facilitate the safe placement of the intended cannula. The needle is thereafter removed from the guide wire, blood vessel and subcutaneous tissue while keeping the guide wire within the vessel. Multiple dilators comprising various outer diameters are inserted over the guide wire and into subcutaneous tissue and the blood vessel in successive steps, from the smallest diameter to the largest, to dilate the vessel to the appropriate size. Afterwards, the dilator is removed, leaving the guide wire within the vessel. The cannula is thereafter placed over the guide wire and into the blood vessel to the desired location therein. The guide wire is thereafter removed from the vessel.

To successively dilate to the appropriate extent, prior art dilators of multiple number and defining various outer diameters from small to large have been utilized, generally measured in units of French gauge or millimeters (1 Fr=0.33 mm). For example, the dilators may comprise increments of French from 14 to 30 (i.e., 14 Fr. to 30 Fr. or 4.62 mm to 9.9 mm). The utilization of prior art dilators is largely successive in their selection. A medical practitioner selects a given dilator that is presumably appropriate for a given blood vessel depending on the size of the cannula, and the dilator is thus inserted into the vessel over the guide wire. If the dilator is too small, it is retracted from the vessel and guide wire and a larger one is thereafter inserted. The foregoing procedure is repeated in succession until the appropriate size dilator is utilized to achieve a target dilation.

However, the foregoing successive use of multiple dilators is fraught with disadvantages. A primary disadvantage in having to successively insert and retract multiple dilators in relation to a given blood vessel is that the successive and repetitive procedure is time consuming. Of course, during a given medical emergency or critical care scenario, time is of the essence; where mere minutes may affect the success of a patient's medical outcome. Furthermore, after a given dilator is inserted into a blood vessel, the medical practitioner cannot see the dilator's location therein, thus requiring the practitioner to imprecisely rely on tactile sensation and anatomical indicators to navigate the dilator to a desired target.

Another disadvantage exists in relation to gripping the dilators. Because the dilators are utilized in relation to blood vessels, their exposure to blood and subcutaneous adipose tissue is inherent, with such blood coating the dilator to make it slippery and thus difficult to grip and manipulate. A further disadvantage exists in relation to the guide wire. This is because the rearward end of the guide wire (i.e., the free end of the wire not located within the patient) typically requires stabilization by a second practitioner while a first practitioner inserts the dilator there-about and into the blood vessel. Without the presence of a second practitioner to stabilize the wire's free rearward end, the end tends to“bounce around” and put the wire at risk of contamination via its possible contact with a non-sterile surface. Also, the wire is at risk of being advanced too far into the patient and lost within the blood vessel as the dilator, located there-around, is being manipulated into the vessel. Furthermore, the wire can also be accidentally pulled back with complete loss of access. This is not only detrimental to the procedure itself, but could lead to serious and life-threatening bleeding complications.

Thus, what is needed is a single dilator having multiple diameters to eliminate or minimize the successive and repetitive use of multiple dilators. The dilator's location within the blood vessel should be readily visible to the medical practitioner to facilitate a ready and accurate placement of the dilator at a targeted location within the vessel. The dilator should also have a handle to facilitate its grip by a medical practitioner while coated with blood and subcutaneous adipose tissue. Furthermore, the dilator should stabilize the free rearward end of the guide wire to prevent the wire from becoming lost within a blood vessel, contaminated, or pulled out. The present invention satisfies these foregoing disadvantages and presents other advantages as well.

### SUMMARY OF THE INVENTION

This invention relates generally to dilators used in endovascular medical procedures. More specifically, the invention relates to an apparatus and method for the dilation of a blood vessel and subcutaneous tissue that eliminates or minimizes a successive and repetitive insertion and removal of multiple, individual dilators necessary to achieve a target dilation.

The dilator comprises a lengthwise structure defining an axial lumen and an outer surface between forward and rearward ends. The lumen is operably engageable with an outer surface of a guide wire while the outer surface of the structure is operably engageable with an inner surface of a blood vessel and subcutaneous tissue. The lumen further defines a forward opening and a rearward opening configured to accept an insertion of a rearward end of the guide wire therein.

The forward end defines an insertion tip configured for insertion into the blood vessel while the outer surface defines at least two cylindrical engagement surfaces of differing outer diameter between the insertion tip and the rearward end, with the at least two cylindrical engagement surfaces separated by a first medial frusto-conical transition surface In a preferred embodiment, the forwardly-located cylindrical engagement surface of the least two cylindrical engagement surfaces has an outer diameter that is smaller than the cylindrical engagement surface located rearwardly thereof. This configuration is crucial in that it facilitates the successive dilation of a blood vessel without having to successively insert and remove multiple dilators to achieve the target dilation. This same configuration is also applicable to any number of cylindrical engagement surfaces defined by the outer surface. Thus, although two such surfaces are discussed herein, it is understood that the outer surface may comprise three, four, or any number of cylindrical engagement surfaces; each separated by further frusto-conical transition surface (i.e., second medial, third medial, etc.).

To assist in navigating the dilator within the blood vessel, the insertion tip and/or each of the at least two cylindrical engagement surfaces preferably define radiopaque markers to make them visible to a medical practitioner via fluoroscopy. These radiopaque markers are preferably distinct from one another such that a given marker indicates the dilator's insertion tip and/or a given engagement surface diameter of the at least two engagement surfaces. Also, a handle is optionally located at the rearward end to enable a medical practitioner to adequately grip and manipulate the dilator despite possibly having a coating of blood or other slippery fluids thereon. The rearward end also optionally defines a stabilizer opening located proximal to the dilator's rearward opening and configured to accept the operable insertion of a rearward end of the guide wire therein. The dilator preferably comprises semi-rigid, medical grade plastic such as polycarbonate, polypropylene, polyethylene and/or custom-made polymers. A hydrophilic coating is optionally located on the exterior surfaces of the dilator to reduce surface friction and enhance lubricity between the dilator and vessel and/or subcutaneous tissue.

While this foregoing description and accompanying figures are illustrative of the present invention, other variations in structure and method are possible without departing from the invention's spirit and scope.

### BRIEF DESCRIPTION OF THE DRAWINGS

FIG. 1 is a perspective view of one embodiment of the dilator with guide-wire;

FIG. 2 is a perspective view of the dilator of FIG. 1 illustrating one embodiment of the lumen;

FIG. 3 is a perspective view of the dilator of FIG. 1 illustrating another embodiment of the lumen;

FIG. 4 is a sectional perspective view of dilator of FIG. 3 better illustrating the lumen;

FIG. 5 is a perspective view of the dilator and wire of FIG. 1 illustrating another embodiment of the insertion tip;

FIG. 6 is a perspective detailed view of a frusto-conical transition surface showing the mathematical relationship between various components;

FIG. 7 is a perspective view of the dilator of FIG. 1 illustrating the fluoroscopy markers;

FIG. 8 is a perspective view of the dilator of FIG. 5 illustrating the fluoroscopy markers;

FIG. 9 is a side elevation detailed view of an embodiment of the optional handle of the dilator; and

FIG. 10 is a perspective detailed view of an embodiment of the optional handle of the dilator having the optional stabilizer opening defined therein.

### DESCRIPTION OF THE EMBODIMENTS

This invention relates generally to dilators used in endovascular medical procedures. More specifically, the invention relates to an apparatus and method for the dilation of a blood vessel and subcutaneous tissue that eliminates or minimizes a successive and repetitive insertion and removal of multiple, individual dilators necessary to achieve a target dilation.

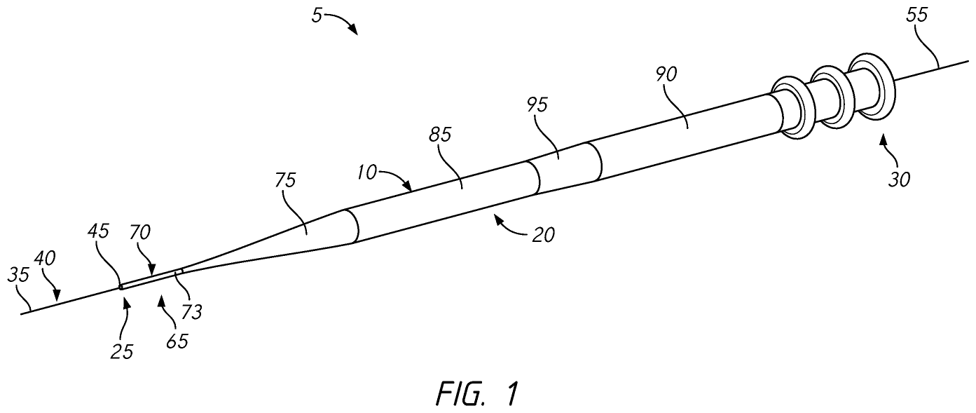

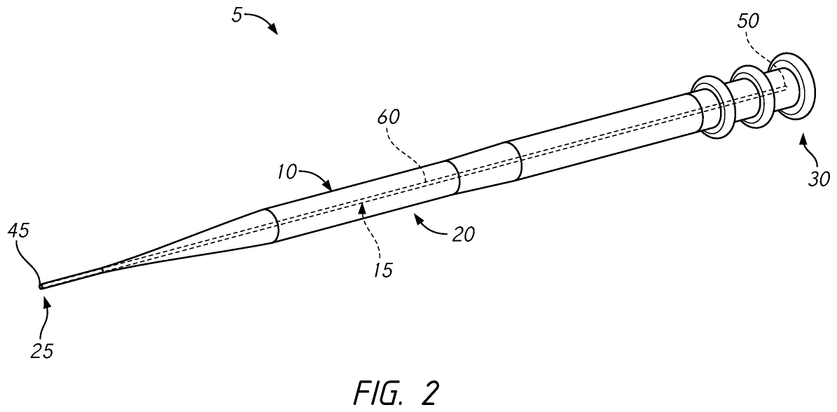

Referring to FIGS. 1 and 2, the dilator 5 comprises a lengthwise structure 10 defining an axial lumen 15 and an outer surface 20 between forward and rearward ends 25 and 30. The lumen 15 is operably engageable with an outer surface 35 of a guide wire 40 while the outer surface 20 of the structure 10 is operably engageable with an inner surface of a blood vessel and subcutaneous tissue (not shown). The lumen 15 further defines a forward opening 45 of the dilator 5 at the forward end 25 of the structure 10 and a rearward opening 50 of the dilator at the structure's rearward end 30. The forward opening 45 is configured to accept an insertion of a rearward end 55 of the guide wire 40 therein to facilitate the operable engagement between the dilator 5 and guide wire.

In the embodiment of FIG. 2, the lumen 15 defines a through cylindrical bore 60 such that both the forward and rearward openings 45 and 50 are circular. The cylindrical bore 60 and forward and rearward openings 45 and 50 define a common diameter that is only slightly larger than the outer diameter of the guide wire 40 such that the dilator 5 can forwardly and rearwardly slide along the guide wire with little or no lateral movement or play occurring between the components. The common diameter of the bore 60 and openings 55 and 60 is between about 0.7 mm and 1.5 mm, more preferably about 1 mm.

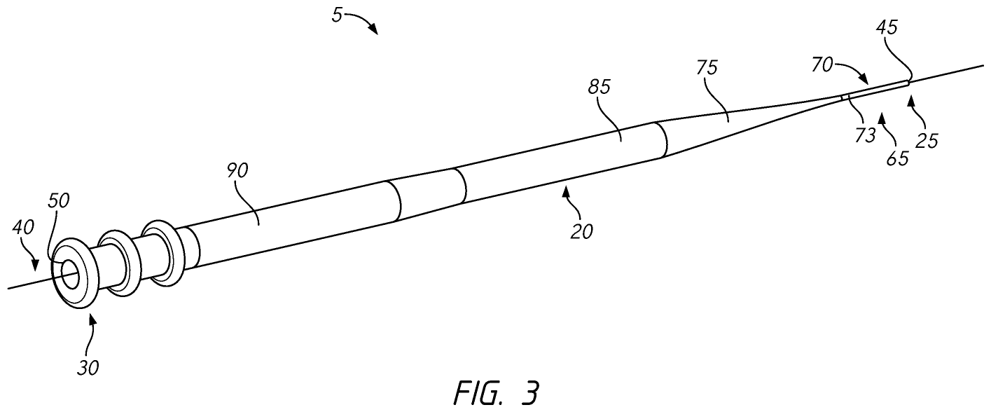

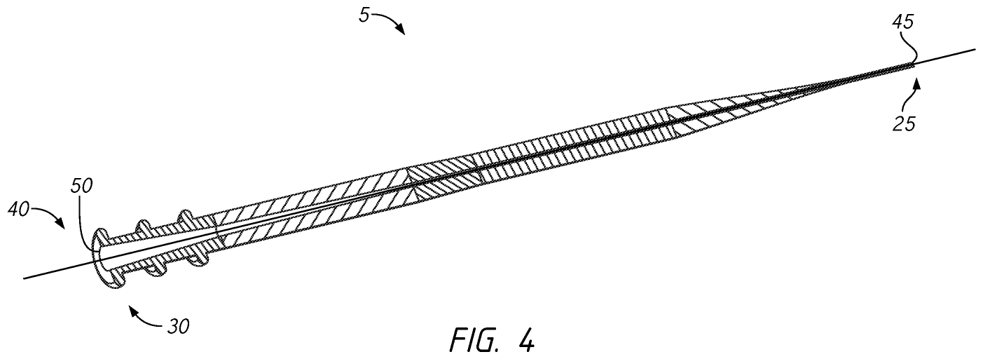

However, in another embodiment illustrated in FIGS. 3 and 4, the rearward opening 50 defines a diameter that is larger than that of the forward opening 45 to allow for lateral movement or play to occur between the guide wire 40 and rearward end 30 of the dilator's structure 10. This lateral movement facilitates an angular adjustment of the dilator 5 in relation to the guide wire 40 to assist in manipulating the dilator into the blood vessel. To define the larger rearward opening 50, the cylindrical lumen 15 gradually increases in diameter towards the rearward end 30. The larger diameter also makes the insertion of the wire 40 easier from the rearward end 30, should this need to occur. Thus, while the diameter of the forward opening 50 is between about 0.7 mm and 1.5 mm and more preferably about 1 mm, the diameter of the rearward opening 50 is between about 3 mm and 8 mm, preferably between about 3.5 and 7 mm, and more preferably about 5 mm.

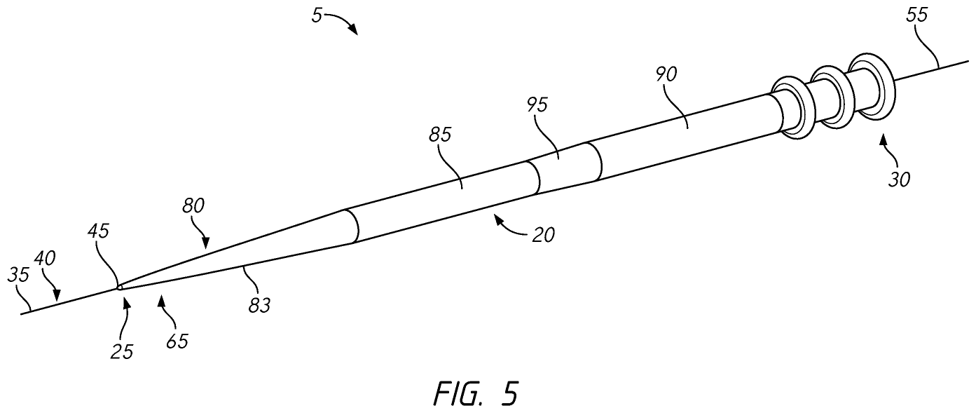

Referring again to FIGS. 1 and 3, the forward end 25 defines an insertion tip 65 located about the forward opening 45 and configured for insertion into the blood vessel. In one embodiment, the insertion tip 65 comprises a hollow cylindrical segment 70 of the structure 10, defining an insertion surface 73 of minimal outside diameter such that the forward end 25 of the dilator 5 can fit within the blood vessel prior to dilating it, to be discussed further. A forward frusto-conical transition surface 75 is located rearwardly of the cylindrical segment 70 of the insertion tip 65 for transitioning to a larger diameter of the outer surface 20 of the remaining structure. In another embodiment, illustrated in FIG. 5, the insertion tip 65 defines a “Coons” tapered segment 80 instead of a cylindrical segment. The Coons tapered segment 80 defines an approximately blunt-ended frusto-conical surface 83 that transitions from the forward opening to, again, a larger diameter of the outer surface 20 of the remaining structure.

Referring to FIGS. 1, 3 and 5, the outer surface 20 of the lengthwise structure 10 defines at least two cylindrical engagement surfaces 85 and 90 of differing outer diameter between the insertion tip 65 and the rearward end 30, with the at least two cylindrical engagement surfaces separated by a first medial frusto-conical transition surface 95. In a preferred embodiment, the forwardly-located cylindrical engagement surface 85 of the at least two cylindrical engagement surfaces 85 and 90 has an outer diameter that is smaller than the cylindrical engagement surface 90 located rearwardly thereof, but larger than the outer diameter of the cylindrical segment 70 of the insertion tip 65 and/or the outer diameter of the Coon's tapered segment 80 at the forward opening 45. This configuration is crucial in that it facilitates the successive dilation of a blood vessel without having to successively insert and remove multiple dilators to achieve the target dilation. This same configuration is also applicable to any number of cylindrical engagement surfaces defined by the outer surface 20. Thus, although two such surfaces 85 and 90 are illustrated and discussed herein, it is understood that the outer surface 20 may comprise three, four, or any number of cylindrical engagement surfaces; each separated by further frusto-conical transition surface (i.e., second medial, third medial, etc.).

To illustrate the value of the foregoing configuration, a further discussion of the inherent disadvantages of the prior art dilators is warranted. When prior art, multiple dilators are utilized, if the first dilator that is threaded over the guide wire and into the blood vessel has an outer diameter that is too small to sufficiently dilate the vessel to a target value, it is withdrawn from the vessel and guide wire, and another dilator of larger outside diameter is threaded over the guide wire and into the vessel in a further attempt to meet the target value. The foregoing procedure is thus repeated, as necessary, until a dilator of sufficient outside diameter is threaded onto the wire and inserted into the vessel to achieve the target dilation. Of course, as briefly discussed within the background portion of this specification, this successive and repetitive dilation procedure is both tedious and dangerously time-consuming during critical care and emergency medical scenarios.

In contradistinction, if the forward cylindrical engagement surface 85 has an outer diameter that is too small to sufficiently dilate the vessel to the target value after applicant's advantageous dilator 5 is threaded over the guide wire 40 and inserted into the blood vessel, the dilator is simply further advanced into the vessel (instead of withdrawing it therefrom) until the next, rearward engagement surface 90 of sufficient outside diameter successfully dilates the vessel to the target value. With further regard to the at least two cylindrical engagement surfaces 85 and 90, their respective outer diameters and lengths, as well as the axial lengths of the frusto conical transition surface(s) 95 located there-between, are of critical importance.

Blood vessels, although flexible, are nonetheless delicate and thus subject to rupture if forced by the outer diameter of a dilator engagement surface that is too large, or if subjected to abrupt transitions from an engagement surface of smaller diameter to one of increased diameter. As such, it is not an advisable practice to directly utilize the largest dilator. Thus, the incremental differences existing in outer diameter from one engagement surface to the next are minimal to ensure that a given successive diameter is not dangerously large for the blood vessel. These engagement surface outer diameters are also integrally related to the axial lengths of the respective frusto-conical transition surfaces located there-between to ensure that only a slight angle of the frusto-conical transition surface is present from one diameter to the next.

Because this angle of the frusto-conical transition surface is geometrically related to the diameters of the associated engagement surfaces, as well as the axial length of the transition surface, a given length of the transition surface is desired to ensure that the transition from one diameter to the next does not occur too abruptly via the utilization of a drastic angle. This same objective applies to the Coon's tapered insertion tip, which transitions from a tip diameter to an engagement wall diameter. Thus, in keeping with the objective of utilizing only a slight angle for the frusto-conical transition surface, a greater incremental diameter difference between forward and rearward engagement surfaces necessitates a longer requisite axial length of the transition surface.

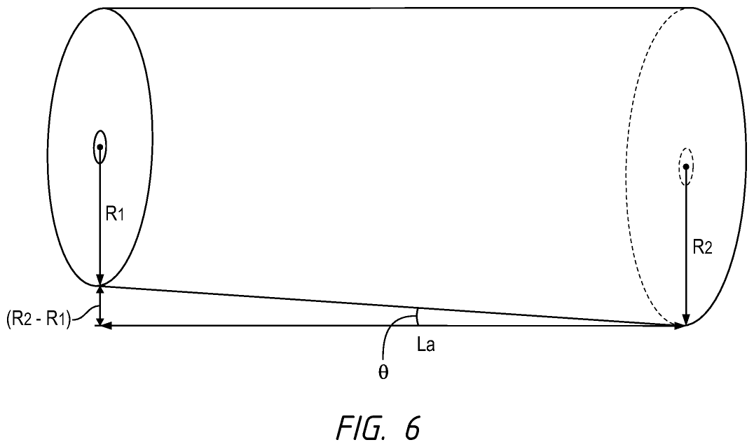

Referring to FIG. 6, the difference in diameter between forward and rearward engagement surfaces, and the axial length and angle of the frusto-conical transition surface, are mathematically related to one another by common trigonometry, namely, tan θ=(R₂−R₁)/La. In the foregoing equation, R₂ is the radius of the (larger) rearward engagement surface, R₁ is the radius of the forward engagement surface, La is the axial length of the frusto-conical transition surface and theta is frusto-conical transition surface's angle from the horizontal.

Furthermore, the lengths of the respective engagement surfaces ensure that an adequate lengthwise portion of the vessel is dilated to sufficiently accommodate the subsequent placement of the cannula therein, while at the same time conserving the overall length of the dilator itself to facilitate its further insertion into the vessel in achieving the target dilation. In other words, if the engagement surfaces are not long enough, the length of the portion of the vessel dilated may be insufficient to accommodate a placement of the cannula therein. If the engagement surfaces are too long, the resulting overall dilator may be too long to allow for its further advancement into the blood vessel to facilitate a usage of an increased-diameter, rearwardly-located engagement surface to achieve the target dilation. Similarly, the length of the insertion tip cylindrical segment is only long enough to ensure that the dilator forward end is adequately inserted into the entrance opening of a given blood vessel while simultaneously preventing it from slipping out of the vessel during placement movements and manipulations.

In embodiments of the dilator illustrated herein, with attention to the foregoing dimension-related objectives, each cylindrical engagement surface 85 and 90 has a length of between about 4 cm and 10 cm, preferably between about 6 cm and 8.5 cm, and more preferably about 6 cm. The forward 85 of the at least two cylindrical engagement surfaces 85 and 90 has an outside diameter of between about 10 Fr. and 26 Fr. (about 3.3 mm and 8.58 mm), while the at least one engagement surface 90 located rearwardly thereof has an outside diameter of between about 14 Fr. and 30 Fr (about 4.62 mm and 9.9 mm). The insertion tip 65 has a cylindrical segment 70 or Coon's taper 80 outside diameter (at the forward end 25) of between about 1 mm and 1.5 mm, more preferably 1.2 mm. The first medial frusto-conical transition surface 95 has a length of between about 1 cm and 4 cm (about 10 mm and 40 mm), more preferably about 2 cm (20 mm), while the forward frusto-conical transition surface 75 or Coon's tapered segment 80 has a length of between about 4 cm and 8 cm, more preferably about 5.5 cm.

While the angle θ of the frusto-conical transition surface should generally not exceed 45 degrees, one may readily calculate various angle values based upon the underlying values of the forward and rearward surface diameters, as well as the axial length of the frusto-conical transition surface. For example, if a frusto-conical transition surface of 20 mm axial length is utilized between forward and rearward engagement surface diameters of 3.3 mm and 9.9 mm, respectively, the frusto-conical transition surface angle θ is readily calculated as:

θ=arc tan((9.9 mm/2)−(3.3 mm/2))/20 mm

θ=arc tan((4.95 mm−1.65 mm)/20 mm

θ=arc tan(3.3 mm/20 mm)

θ=arc tan(0.165 mm)

θ=9.37 degrees

Of course, if an optimum angle θ is known, one can also calculate optimum values for an engagement surface diameter or frusto-conical transition surface axial length, so long as other necessary values are known.

To assist in navigating the dilator within the blood vessel, the insertion tip 65 and/or each of the at least two cylindrical engagement surfaces 85 and 90 preferably define radiopaque markers to make them visible to a medical practitioner via fluoroscopy. These radiopaque markers are preferably distinct from one another such that a given marker indicates the dilator's insertion tip 65 and/or a given engagement surface diameter of the at least two engagement surfaces 85 and 90. The distinctness of the markers from one another is achieved through the use of radiopaque compounds defining differing fluoroscopy contrasts, with such differing contrasts achieved via a use of differing radiopaque compounds and/or their respective differing volumetric percentages or patterns utilized within the marker.

The radiopaque compounds may comprise barium sulfate, bismuth trioxide, bismuth oxychloride, bismuth subcarbonate, tungsten or other materials known in the art as possessing radiopaque properties. The compounds may be formulated into respective coatings for application to the external surfaces of the insertion tip and/or respective engagement surfaces, or mixed directly into the medical grade plastic or polymer materials utilized in manufacturing the respective tip and/or engagement surfaces themselves.

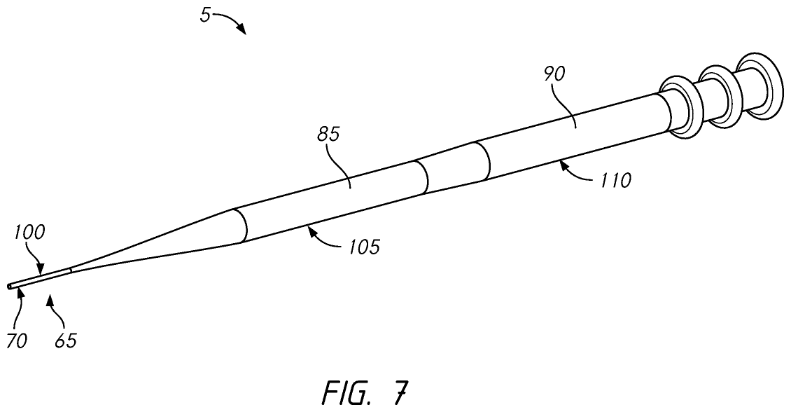

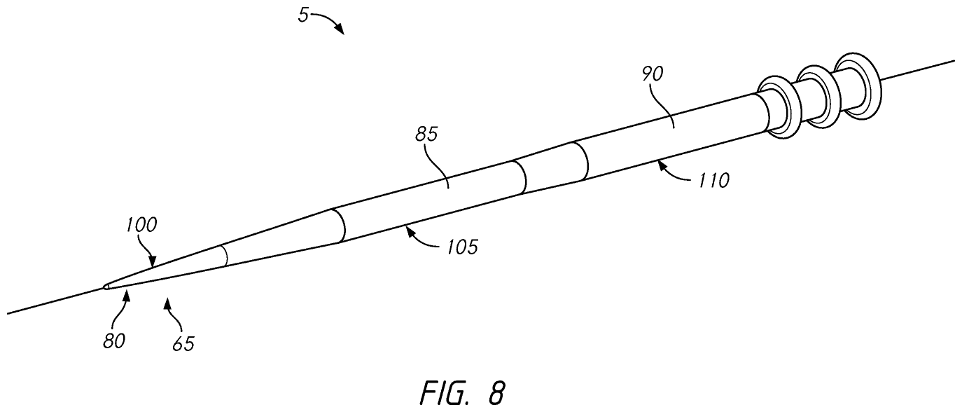

FIGS. 7 and 8 illustrate embodiments of the dilator 5 defining radiopaque markers. The respective markers 100, 105 and 110 of the insertion tip 65 and/or the at least two cylindrical engagement surfaces 85 and 90 each define a distinct, predetermined radiopaque contrast such that the medical practitioner can readily distinguish these component from one another via fluoroscopy while the dilator is located within the blood vessel. The markers may be standardized to a given component and/or diameter to enable a practitioner to readily identify each under fluoroscopy. For example, the marker 100 of the smaller-diameter insertion tip 65 of the dilator 5, to include the cylindrical segment 70 (FIG. 7) and Coons tapered segment 80 at the forward end 25 (FIG. 8), may have a fluoroscopy contrast (i.e., using 40% by volume of barium sulphate as the radiopaque material) that is higher than the marker 105 of the forwardly-located, larger-diameter engagement surface 85 (i.e., which uses 30% by volume of barium sulphate as the radiopaque material), which in turn is higher than the marker 110 of the rearwardly-located, yet larger-diameter engagement surface 90 (i.e., which uses only 20% by volume of barium sulphate as the radiopaque material).

In the foregoing embodiment, the decrease in percentage by volume of radiopaque material utilized for the marker of a given component is preferably inversely proportional to that component's diameter to ensure that a component of smaller diameter possesses a higher fluoroscopy contrast (i.e., a higher visibility) than a component of larger diameter. This relationship is crucial in ensuring that the smaller components of the dilator have a proportionately greater fluoroscopic visibility to the medical practitioner during navigation and placement of the dilator with the blood vessel. The foregoing relationship thus readily facilitates: the visible identification of the smaller-diameter, but high-contrast insertion tip 65 and its location and depth within the blood vessel; the visible identification of the larger-diameter, but medium-contrast forwardly-located engagement surface 85 within the target dilation area of blood vessel; and the visible identification of the yet larger-diameter, but low-contrast rearwardly-located engagement surface 90 within the blood vessel, if necessitated by the target dilation area.

Although the varying marker contrasts of the foregoing example utilizes the varying percentage by volume of a common radiopaque material (i.e., barium sulphate), it is understood that these varying contrasts of the markers may be achieved by utilizing different radiopaque materials possessing differing contrast values (i.e., bismuth subcarbonate possessing a greater contrast value than bismuth trioxide, which in turn has a greater contrast value than barium sulphate), or by utilizing various combinations of both differing materials and differing percentage by volume quantities. Furthermore, the marker contrasts may have other relationships as well, to include those that are proportional to a given diameter, equal to one another, or any other relation understood in the art as providing indicating value to a medical practitioner.

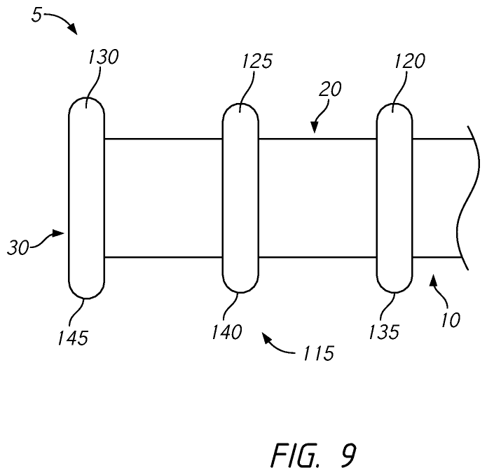

As illustrated in the figures, a handle 115 is optionally located at the rearward end 30 of the structure. The handle 115 enables a medical practitioner to adequately grip and manipulate the dilator 5 despite the dilator possibly having a coating of blood or other slippery fluids thereon. Referring to FIG. 9, the handle 115 comprises a plurality of spatially-arranged toroids (i.e., 120, 125 and 130) extending outwardly from the structure's outer surface 20. Their extension from the outer surface, each thus defining a diameter exceeding that of any engagement surface (i.e., of engagement surfaces 85 and 90), ensures that the dilator 5 does not become inserted too deeply into the blood vessel, thus possibly losing the dilator's rearward end within the vessel's entrance opening. Because the outer diameters of the toroids presumably exceed the inner diameter of the blood vessel's entrance opening, their occlusion at the opening prevents the foregoing scenario from occurring.

Additionally, the outer radial ends 135, 140 and 145 respectively defined by the toroids 120, 125 and 130 each present a smooth, rounded surface for a medical practitioner to grip without the risk of tearing sterile gloves comprised of latex or similar delicate materials. Although three toroids 120, 125 and 130 are illustrated within the figures, it is nonetheless understood that additional or fewer toroids may be utilized as well. Furthermore, although the figures illustrate that the rearward-most toroid 130 (i.e., that which is co-terminus with the rearward end 30 of the structure 10) defines an outer diameter exceeding that that commonly defined by the remaining toroids 120 and 125, it is further understood that the plurality of toroids may each define a common diameter, or diameters that are different from one another as well.

As such, the rearward-most toroid 130 defines an outer diameter of between about 1 and 3 cm, more preferably between 1 and 1.75 cm, while the remaining toroids 120 and 125 of the plurality each define an outside diameter of between about 0.75 and 2 cm, more preferably between 0.75 and 1.6 cm. Each toroid 120, 125 and 130 defines a wall-to-wall width of between about 2 and 7 mm, more preferably between 3 and 5 mm, and is spatially separated from one another via a wall-to-to wall distance of between about 7 and 14 cm, more preferably between 9 and 12 cm.

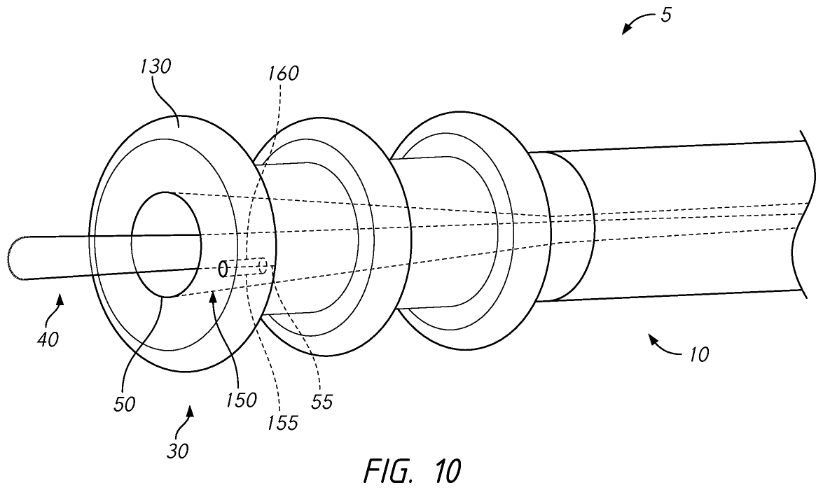

In yet another embodiment illustrated in FIG. 10, the rearward end 30 of the structure 10 defines a stabilizer opening 150 located proximal to the dilator's rearward opening 50. The stabilizer opening preferably 150 comprises a small, through bore 155 defined through the rear-most toroid 130, and is configured to accept the operable insertion of the rearward end 55 of the guide wire 40 therein. The bore 155 is preferably parallel to the axial lumen 15 and defines a diameter slightly larger than that of the guide wire's outer diameter such that the end 55 is secured within the bore via a friction fit. After the dilator 5 is threaded onto the guide wire 40, the rearward end 55 of the wire is “bent-around” in a forwardly direction and the end is inserted into and through the bore 155 of the stabilizer opening 150.

The friction fit, created by the spring force of the bent guide wire 40 acting against the interior surface 160 of the bore 155 while the end 55 of wire is inserted there-through, prevents the end of the wire from pulling out or otherwise becoming loose again. Also, the frictional securement of the guide wire's rearward end 55 within the stabilizer opening 150 prevents the guide wire 40 from becoming lost within a blood vessel. Furthermore, the securement of the guide wire's rearward end 55 within the stabilizer opening also prevents the end from possible contamination via a contact that end with a possibly non-sterile surface.

The bore 155 of the stabilizer opening 150 preferably defines a diameter of between about 1 mm and 1.5 mm, more preferably 1 mm to ensure that it is slightly larger than the outer diameter of a common guide wire. In a preferred embodiment of the invention, the stabilizer and rearward openings 150 and 50 are axially parallel with one another, with the stabilizer opening being radially displaced from the rearward opening by an axially center-to-center distance of between about 2 and 10 mm, more preferably by between 3.5 and 8 mm.

In a preferred embodiment of the invention, the dilator 5 comprises semi-rigid, medical grade plastic such as polycarbonate, polypropylene, polyethylene and/or custom-made polymers, with all of the components of the dilator being unitary with one another as a result of its underlying manufacture via a precise plastic injection molding process. The semi-rigidity of the plastic allows the dilator 5 to remain flexible along its length while simultaneously remaining rigid enough for its outer surface 20 to exert suitable pressure against the inner surface of the blood vessel. It is understood, however, that the dilator and all of its underlying components may comprise rigid plastic, as well as aluminum and other similar materials. Additional embodiments utilize a hydrophilic coating (not shown) on the exterior surfaces of the dilator 5 to reduce surface friction and enhance lubricity between the dilator and vessel and/or subcutaneous tissue.

In use, a scalpel is used, if necessary, to perform a tissue cut-down to expose the target blood vessel. The vessel is thereafter punctured with a needle. The forward end of a guide wire is thereafter inserted through the needle and into the vessel to be dilated, and advanced forwardly by an appropriate distance to allow for the safe placement of the intended cannula. The needle is thereafter removed from the guide wire and vessel, leaving the guide wire within the vessel.

The dilator is thereafter threaded onto the guide wire via an insertion of the wire's rearward end into the dilator's forward opening. After the dilator is advanced (i.e., slid) forwardly along the guide wire until the wire's rearward end becomes accessible through the dilator's rearward opening, the free end of the wire is optionally bent around and inserted into the dilator's stabilizer opening, remaining therein via a friction fit existing between the components.

With the optional use of a fluoroscope, the insertion tip of the dilator is then inserted into the blood vessel through the vessel's opening created by the needle. The insertion tip may include a radiopaque marker to make the tip visible to the medical practitioner, via the fluoroscope, while inserted within the vessel. While gripping the optional handle, the dilator is sufficiently advanced in a forward direction such that a forwardly-located cylindrical engagement surface of the at least two engagement surfaces, each optionally including respective contrasting radiopaque markers, is advanced into the vessel with the optional aid of the fluoroscope, with the target dilation of the vessel thereafter assessed.

If the target dilation is not achieved, the dilator is further advanced into the vessel until the first medial frusto-conical transition surface and next rearwardly-located cylindrical engagement surface proceeds through the vessel's opening and into the vessel, again with the optional aid of the fluoroscope and radiopaque marker, with the target dilation of the vessel again assessed. The foregoing advancement of the dilator and successive transition and cylindrical engagement surfaces is repeated, again with the optional aid of a fluoroscope and respective radiopaque markers, until the target dilation of the vessel is achieved.

After the target vessel is dilated to the appropriate target size via the operable engagement of the appropriate engagement surface with the vessel, the rearward end of the guide wire is removed from the stabilization opening of the dilator, if optionally used during the procedure, and the dilator is removed from the vessel and guide wire. The desired cannula is thereafter threaded over the guide wire and through the opening of the blood vessel and into the now-dilated vessel by an appropriate distance.

While this foregoing description and accompanying figures are illustrative of the present invention, other variations in structure and method are possible without departing from the invention's spirit and scope.

We claim:

1. A dilator for a blood vessel and subcutaneous tissue comprising:
a lengthwise structure defining a lumen and an outer surface between forward and rearward ends, the lumen operably engageable with an outer surface of a guide wire and the outer surface of the lumen operably engageable with an inner surface of the blood vessel, the lumen defining a forward opening at the forward end and a rearward opening at the rearward end, the forward end defining an insertion tip located about the forward opening and configured for insertion into the blood vessel, the forward opening configured to accept an insertion of a rearward end of the guide wire therein, the outer surface defining at least two cylindrical engagement surfaces of differing diameter between the insertion tip and the rearward end, the at least two cylindrical engagement surfaces separated by a frusto-conical transition surface.

2. The dilator of claim 1 wherein the rearward end defines a stabilizer opening located proximal to the rearward opening, the stabilizer opening configured to accept an insertion of a rearward end of the guide wire therein.

3. The dilator of claim 2 wherein the rearward end further defines a handle.

4. The dilator of claim 1 wherein one of the at least two cylindrical contact surfaces defines an outer diameter of between about 10 and 26 Fr and the other of the at least two cylindrical contact surfaces defines an outer diameter of between about 14 and 30 Fr.

5. The dilator of claim 4 wherein the at least two cylindrical contact surfaces each define a length of between about 6 and 8.5 cm.

6. The dilator of claim 5 wherein the first medial frusto-conical transition surface defines a length of between about 1 and 4 cm and the forward frusto-conical transition surface defines a length of between 4 and 8 cm.

7. The dilator of claim 6 wherein the lumen is comprised of semi-rigid medical grade plastic.

8. The dilator of claim 1 wherein the insertion tip is selected from a group consisting of a cylindrical segment and a coons tapered segment.

9. The dilator of claim 8 wherein the lumen defines a cylindrical bore, and the forward and rearward openings are circular and define a common diameter.

10. The dilator of claim 8 wherein the forward and rearward openings are circular, the rearward opening defines a diameter larger than a diameter defined by the forward opening, and the lumen defines a cylindrical bore at the forward end that gradually increases in diameter towards the rearward end.

11. The dilator of claim 1 wherein the insertion tip defines a radiopaque marker.

12. The dilator of claim 11 wherein each of the at least two engagement surfaces defines radiopaque markers.

13. The dilator of claim 12 wherein the radiopaque markers defined by the insertion tip and at least two engagement surfaces each define respectively different contrasts.

14. The dilator of claim 1 wherein a hydrophilic coating is located on the outer surface.

15. A method of dilating a blood vessel comprising:
threading a dilator onto a guide wire located within the vessel;
inserting an insertion tip of the dilator into the vessel;
advancing a forward engagement surface of at least two engagement surfaces defined by the dilator into the vessel to achieve a target dilation;
assessing the vessel to determine if the target dilation has been achieved by the forward engagement surface;
advancing a rearward engagement surface of the at least two engagement surfaces into the vessel to achieve the target dilation if said target dilation has not been achieved by the forward engagement surface;
assessing the vessel to determine if the target dilation has been achieved by the rearward engagement surface; and
withdrawing the dilator from the vessel if the target dilation has been achieved by the rearward engagement surface.

16. The method of claim 15 wherein the insertion tip is selected from a group consisting of a cylindrical segment and a coons tapered segment.

17. The method of claim 15 further comprising viewing with a fluoroscope a radiopaque marker defined by the insertion tip.

18. The method of claim 17 further comprising viewing with a fluoroscope radiopaque markers respectively defined by each of the forward and rearward engagement surfaces of the at least two engagement surface.

19. The method of claim 19 further comprising viewing with a fluoroscope differing contrasts respectively defined by each of said markers.

20. The method of claim 15 further comprising locating a hydrophilic coating on the dilator prior to inserting the insertion tip into the vessel.
